(19) United States
(12) Patent Application Publication
(10) Pub. No.: US 2006/0079552 A1
(43) Pub. Date: Apr. 13, 2006

(54) 1-[2-(4-benzyl-4-hydroxy-piperidin-1-yl)-ethyl]-3-(2-methyl-quinolin-4-yl)-urea salt

(76) Inventors: Jorg Velker, Lorrach (DE); Michael Scherz, Ettingen (CH); Thomas Weller, Binningen (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/962,960

(22) Filed: Oct. 12, 2004

(51) Int. Cl. A61K 31/4709; C07D 401/02

(52) U.S. Cl. 514/313; 546/159

(57) ABSTRACT

The invention relates to 1-[2-(4-benzyl-4-hydroxy-piperidin-1-yl)-ethyl]-3-(2-methyl-quinolin-4-yl)-urea as a crystalline salt or a non defined salt hydrate thereof and a process for its preparation. Further, the present invention relates to the use of said 1-[2-(4-benzyl-4-hydroxy-piperidin-1-yl)-ethyl]-3-(2-methyl-quinolin-4-yl)-urea as a crystalline salt alone or in combination with other compounds or formulations of said 1-[2-(4-benzyl-4-hydroxy-piperidin-1-yl)-ethyl]-3-(2-methyl-quinolin-4-yl)-urea as a crystalline salt in the preparation of pharmaceutical compositions. The invention also relates to the use of such salts in formulations as neurohormonal antagonists.

The present invention relates to 1-[2-(4-benzyl-4-hydroxy-piperidin-1-yl)-ethyl]-3-(2-methyl-quinolin4-yl)-urea as a crystalline salt or a non-defined salt hydrate thereof and a process for its preparation. Further, the present invention relates to the use of said 1-[2-(4-benzyl-4-hydroxy-piperidin- 1-yl)-ethyl]-3-(2-methyl-quinolin-4-yl)-urea as a crystalline salt alone or in combination with other compounds. The present invention also relates to compositions containing said 1-[2-(4-benzyl-4-hydroxy-piperidin-1-yl)-ethyl]-3-(2-methyl-quinolin-4-yl)-urea as a crystalline salt and inert carrier material which are useful as urotensin-II antagonist.

1-[2-(4-Benzyl-4-hydroxy-piperidin-1-yl)-ethyl]-3-(2-methyl-quinolin4-yl)-urea of formula I as well as the process for its preparation as free base is known from WO-2004026836. 1-[2-(4-Benzyl-4-hydroxy-piperidin-1-yl)-ethyl]-3-(2-methyl-quinolin-4-yl)-urea of formula I has been shown to be a potent urotensin II receptor antagonist [Martine Clozel et al. in J. Pharmcol. Exp. Ther. 2004, 311, 204-212].

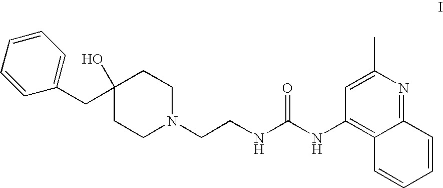

1-[2-(4-Benzyl-4-hydroxy-piperidin-1-yl)-ethyl]-3-(2-methyl-quinolin-4-yl)-urea of formula I as free base has the disadvantages that it is hygroscopic, its colour changes at higher temperature and higher humidity, and it agglomerates to a substance cake under these conditions. 1-[2-(4-Benzyl-4-hydroxy-piperidin-1-yl)-ethyl]-3-(2-methyl-quinolin-4-yl)-urea of formula I as free base is slightly soluble in water at pH 7 (compare Example 9). The said compound of formula I as free base was shown to have a low bioavailability after oral dosing in the rat (compare Example 10). Therefore, the said compound of formula I as free base is not suitable as a pharmaceutical product since it is not easy to handle in pharmaceutical preparations. In addition, large scale production and storage of the said compound of formula I causes problems due to the properties mentioned above.

The subject of the present invention is to provide 1-[2-(4-benzyl-4-hydroxy-piperidin-1-yl)-ethyl]-3-(2-methyl-quinolin-4-yl)-urea of formula I in crystalline forms which show improved properties suitable for a pharmaceutical product, pharmaceutical preparations, production in large scale, and storage.

The present invention relates to a compound, 1-[2-(4-benzyl-4-hydroxy-piperidin-1-yl)-ethyl]-3-(2-methyl-quinolin-4-yl)-urea of formula I as a sulfate or non-defined sulfate hydrate as shown below. 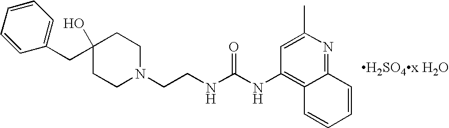
wherein x is 0 or larger.

A sulfate salt of 1-[2-(4-benzyl-4-hydroxy-piperidin-1-yl)-ethyl]-3-(2-methyl-quinolin-4-yl)-urea of formula I is described by [Martine Clozel et. al., J Pharmacol Exp Ther. 2004; DOI:10.1124/jpet.104.068320] but no procedure for its preparation has been disclosed.

The present invention in addition also relates to a compound, 1-[2-(4-benzyl-4-hydroxy-piperidin-1-yl)-ethyl]-3-(2-methyl-quinolin-4-yl)-urea of formula I as a malate or non-defined malate hydrate as shown below. 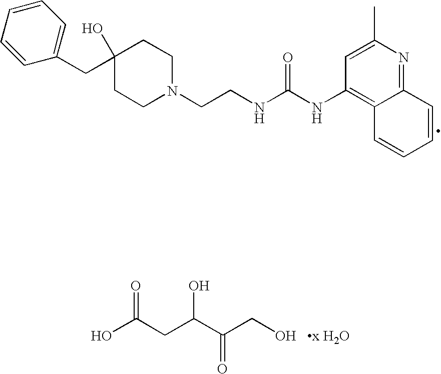
wherein x is 0 or larger.

Further the present invention also relates to a compound, 1-[2-(4-benzyl-4-hydroxy-piperidin-1-yl)-ethyl]-3-(2-methyl-quinolin-4-yl)-urea of formula I as a citrate or non-defined citrate hydrate as shown below. 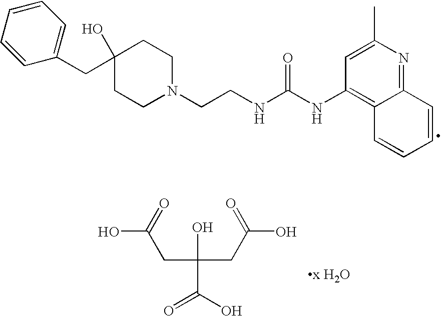
wherein x is 0 or larger.

The present invention also relates to a process for preparing the above mentioned salts of 1-[2-(4-benzyl-4-hydroxy-piperidin-1-yl)-ethyl]-3-(2-methyl-quinolin-4-yl)-urea which process comprises
a) mixing 1-[2-(4-benzyl-4-hydroxy-piperidin-1-yl )-ethyl]-3-(2-methyl-quinolin-4-yl)-urea of formula I with an organic solvent and adding an acid, a solution of an acid in water, a solution of an acid in an organic solvent, or a solution of an acid in a mixture of water and an organic solvent, and stirring the mixture; or
b) mixing 1-[2-(4-benzyl-4-hydroxy-piperidin-1-yl )-ethyl]-3-(2-methyl-quinolin-4-yl)-urea of formula I with a mixture of an organic solvent and water and adding an acid, a solution of an acid in water, a solution of an acid in an organic solvent, or a solution of an acid in a mixture of water and an organic solvent, and stirring the mixture; or
c) adding 1-[2-(4-benzyl-4-hydroxy-piperidin-1-yl)-ethyl]-3-(2-methyl-quinolin-4-yl)-urea of formula I as a solid, or dissolved in a mixture of an organic solvent and water to an acid, to a solution of an acid in water, to a solution of an acid in an organic solvent, or to a solution of an acid in a mixture of water with an organic solvent, and stirring the mixture; or
d) adding 1-[2-(4-benzyl-4-hydroxy-piperidin-1-yl)-ethyl]-3-(2-methyl-quinolin-4-yl)-urea of formula I as a solid, or dissolved in an organic solvent to an acid, to a solution of an acid in water, to a solution of an acid in an organic solvent, or to a solution of an acid in a mixture of water with an organic solvent, and stirring the mixture.

The acids used in the above process are sulfuric acid, malic acid, and citric acid (compare also Examples 1 to 6).

Further, the present invention relates to 1-[2-(4-benzyl-4-hydroxy-piperidin-1-yl)-ethyl]-3-(2-methyl-quinolin-4-yl)-urea salts obtainable by the process mentioned above.

Further, the present invention relates to pharmaceutical compositions comprising 1-[2-(4-benzyl-4-hydroxy-piperidin-1-yl)-ethyl]-3-(2-methyl-quinolin-4-yl )-urea salts as mentioned above and inert carrier material.

Further, the present invention relates to 1-[2-(4-benzyl-4-hydroxy-piperidin-1-yl)-ethyl]-3-(2-methyl-quinolin-4-yl)-urea salts as mentioned above and their use as medicaments.

Because of their ability to inhibit the actions of urotensin II, 1-[2-(4-benzyl-4-hydroxy-piperidin-1-yl)-ethyl]-3-(2-methyl-quinolin-4-yl)-urea salts as described above can be used for treatment of diseases which are associated with an increase in vasoconstriction, proliferation or other disease states associated with the actions of urotensin II. Examples of such diseases are hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, or pulmonary fibrosis. They can also be used for prevention of restenosis after balloon or stent angioplasty, for the treatment of cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, therapy and prophylaxis of diabetic complications, complications of vascular or cardiac surgery or after organ transplantation, complications of cyclosporin treatment, pain, addictions, schizophrenia, Alzheimer's disease, anxiety, obsessive-compulsive behavior, epileptic seizures, stress, depression, dementias, neuromuscular disorders, neurodegenerative diseases, as well as other diseases related to a dysregulation of urotensin II or urotensin II receptors.

These compositions may be administered in enteral or oral form e.g. as tablets, dragees, gelatine capsules, emulsions, solutions or suspensions, in nasal form like sprays and aerosols, or rectally in form of suppositories. 1-[2-(4-Benzyl-4-hydroxy-piperidin-1-yl)-ethyl]-3-(2-methyl-quinolin-4-yl)-urea salts as mentioned above may also be administered in intramuscular, parenteral or intravenous form, e.g. in form of injectable solutions.

These pharmaceutical compositions may contain 1-[2-(4-benzyl-4-hydroxy-piperidin-1-yl)-ethyl]-3-(2-methyl-quinolin-4-yl)-urea salts as mentioned above in combination with inorganic and/or organic excipients, which are usual in the pharmaceutical industry, like lactose, maize or derivatives thereof, talcum, stearic acid or salts of these materials.

For gelatine capsules vegetable oils, waxes, fats, liquid or half-liquid polyols etc. may be used. For the preparation of solutions and sirups e.g. water, polyols, saccharose, glucose etc. are used. Injectables are prepared by using e.g. water, polyols, alcohols, glycerin, vegetable oils, lecithin, liposomes etc. Suppositories are prepared by using natural or hydrogenated oils, waxes, fatty acids (fats ), liquid or half-liquid polyols etc.

The compositions may contain in addition preservatives, stabilisation improving substances, viscosity improving or regulating substances, solubility improving substances, sweeteners, dyes, taste improving compounds, salts to change the osmotic pressure, buffer, anti-oxidants etc.

1-[2-(4-Benzyl-4-hydroxy-piperidin-1-yl)-ethyl]-3-(2-methyl-quinolin-4-yl)-urea salts as mentioned above may also be used in combination with one or more other therapeutically useful substances e.g. with α- and β-blockers like phentolamine, phenoxybenzamine, atenolol, propranolol, timolol, metoprolol, carteolol, carvedilol, etc.; with vasodilators like hydralazine, minoxidil, diazoxide, flosequinan, etc.; with calcium-antagonists like diltiazem, nicardipine, nimodipine, verapamil, nifedipine, etc.; with angiotensin converting enzyme-inhibitors like cilazapril, captopril, enalapril, lisinopril etc.; with potassium channel activators like pinacidil, chromakalim, etc.; with angiotensin receptor antagonists like losartan, valsartan, candesartan, irbesartan, eprosartan, telmisartan, and tasosartan, etc.; with diuretics like hydrochlorothiazide, chlorothiazide, acetolamide, bumetanide, furosemide, metolazone, chlortalidone, etc.; with sympatholytics like methyidopa, clonidine, guanabenz, reserpine, etc.; with endothelin receptor antagonists like bosentan, tezosentan, clazosentan, darusentan, atrasentan, enrasentan, or sitaxsentan, etc.; with anti-hyperlipidemic agents like lovastatin, pravastatin, fluvastatin, atorvastatin, cerivastatin, simvastatin, etc.; and other therapeutics which serve to treat high blood pressure, vascular disease or other disorders listed above.

The dosage may vary within wide limits but should be adapted to the specific situation. In general the dosage given daily in oral form should be between about 3 mg and about 3 g, preferably between about 5 mg and about 1 g, especially preferred between 10 mg and 300 mg, per adult with a body weight of about 70 kg. The dosage should be administered preferably in 1 to 3 doses of equal weight per day. As usual children should receive lower doses which are adapted to body weight and age.

The present invention also relates to compositions containing amorphous parts of 1-[2-(4-benzyl-4-hydroxy-piperidin-1-yl)-ethyl]-3-(2-methyl-quinolin-4-yl)-urea salts as mentioned above.

The term “crystallinity” or “crystalline” is used to describe the part of crystalline material compared to amorphous material and is estimated e.g. by the line shape and the background intensity in X-ray diffraction patterns.

According to these methods, a crystallinity of 90% to 100% is estimated. In a more preferred embodiment the crystallinity is within the range of 92% to 100%. In the most preferred embodiment the crystallinity is within the range of 95% to 100%.

The term “non-defined crystalline salt hydrate” is used to describe salts that contain variable amounts of water. A part or all of the water molecules can be bound the crystal lattice. The term “non-defined crystalline salt hydrate” also describes salts that contain water that is not bound to the crystal lattice. The amount of water contained in a “non-defined crystalline salt hydrate” is within a range of 0 to 20%, preferably within a range of 0 to 10%.

The term “non-defined sulfate hydrate” is used to describe 1-[2-(4-benzyl-4-hydroxy-piperidin-1-yl)-ethyl]-3-(2-methyl-quinolin-4-yl )-urea sulfate salts that contain variable amounts of water as described above.

The term “non-defined malate hydrate” is used to describe 1-[2-(4-benzyl-4-hydroxy-piperidin-1-yl)-ethyl]-3-(2-methyl-quinolin-4-yl)-urea malate salts that contain variable amounts of water as described above.

The term “non-defined citrate hydrate” is used to describe 1-[2-(4-benzyl-4-hydroxy-piperidin-1-yl)-ethyl]-3-(2-methyl-quinolin-4-yl)-urea citrate salts that contain variable amounts of water as described above.

The term “acid”, as used within the present invention, means acids, such as sulfuric acid, malic acid, and citric acid. Malic acid like all optically active acids can be used as a racemate, as optically pure enantiomer, and mixtures of enantiomers. Especially preferred acids are sulfuric acid, and malic acid. Most preferred acid is sulfuric acid. The acid may be used without solvent or dissolved either in organic solvents, mixtures of organic solvents and water, or water. Preferably, the acid is dissolved in mixtures of organic solvents and water, or in water.

The term “organic solvents”, as used within the present invention, means solvents or mixtures of solvents, such as C₁₋₄-alkanol (CH₃OH, C₂H₅OH, n-C₃H₇OH, i-C₃H₇OH, n-C₄H₉OH, i-C₄H₉OH, t-C₄H₉OH), ketones (acetone, ethylmethylketone, methylisobutylketone), ethers (diethylether, tetrahydrofurane, 1,4-dioxane, methyl-tert.butylether) or acetonitrile. Preferred “organic solvents” are CH₃OH, C₂H₅OH, n-C₃H₇OH, i-C₃H₇OH and acetone. Most preferred “organic solvents” are CH₃OH, C₂H₅OH, i-C₃H₇OH and acetone.

The term “solution of an acid” as used within the present invention, means solutions of an acid as described before, preferably aqueous solutions. Acid solutions are in the concentration range of 0.01 to 10 mol/L, more preferred in the concentration range of 0.1 to 5 mol/L, most preferred in the concentration range of 0.5 to 2 mol/L.

The foregoing general description of the invention will now be further illustrated with a number of non-limiting examples.

### EXAMPLES OF THE INVENTION

### LIST OF ABBREVIATIONS

aq. aqueous
AUC area under the curve
DMSO dimethylsulfoxide
HV high vacuum conditions
J coupling constant in NMR
min minutes
MHz megahertz
MP melting point
NMR nuclear magnetic resonance
ppm part per million
RH relative humidity
r.t. room temperature
XRD X-ray powder diffraction

NMR spectra were recorded on a Varian Mercury 300VX NMR Spectrometer. The spectra are referenced to tetramethylsilane as external standard. X-ray diffraction patterns (XRD) were recorded on a Bruker D5000, using a CU-Kₐₗₚₕₐ (1.5418 Å) source, a 40 kV-30 mA generator, in a range of 3 et 40° (2theta). Stress test studies were done by exposing samples in open and closed glass bottles to the following conditions: 60° C./80% RH (8 weeks) and 80° C./RH not controlled (48 h).

### Example 1

### 1-[2-(4-Benzyl-4-hydroxy-piperidin-1-yl)-ethyl]-3-(2-methyl-quinolin-4-yl)-urea sulfate

To a suspension of 1-[2-(4-benzyl-4-hydroxy-piperidin-1-yl)-ethyl]-3-(2-methyl-quinolin-4-yl)-urea of formula I (9.70 g, 0.228 mol) in CH₃OH (250 mL, 0.9 M solution of compound 1) is added aqueous H₂SO₄ (11.4 mL, 2 M, 0.228 mol). The clear solution is stirred at 4° C. for 15 h. The formed precipitate is filtered, washed with CH₃OH (2×10 mL) and dried in HV to provide the title compound as white crystalline powder in 83% yield.

Analytics

MP: 239-242° C. (decomposition).

H₂O content: 1.41%.

Elemental Analysis for C₂₅H₃₂N₄O₆S (0.41 H2O): % found (calculated): C: 57.20 (57.30); H 6.37 (6.31); N 10.73 (10.69); S 6.14 (6.12).

¹H-NMR (d6-DMSO): 8.26 (d, J=8.5, 1H); 8.09 (s, 1H); 7.83 (d, J=8.2, 1H); 7.70 (t, J≈7.6, 1H); 7.51 (t, J≈7.6, 1H); 7.43 (br. s,1 H); 7.27-7.15 (m, 5H); 4.77 (br. s, 1 H); 3.54-3.53 (m, 2H); 3.35-3.31 (m, 2H); 3.20-3.06 (m, 5H); 2.70 (s, 2H); 2.58 (s, 3H); 1.83-1.75 (m, 2H); 1.59-1.54 (m, 2H).

### Example 2

### 1-[2-(4-Benzyl-4-hydroxy-piperidin-1-yl)-ethyl]-3-(2-methyl-quinolin-4-yl)-urea sulfate

To a suspension of 1-[2-(4-benzyl-4-hydroxy-piperidin-1-yl)-ethyl]-3-(2-methyl-quinolin-4-yl)-urea of formula I (4.3 kg) in CH₃OH (86 L) is added aqueous H₂SO₄ (8.5 L, 9.91%) during 15 min. The solution is cooled to −8° C. and stirred at this temperature for 1 h. The formed precipitate is filtered, washed with cooled CH₃OH (−5° C., 2×9 L) and dried under a stream of nitrogen to provide the title compound as white crystalline powder in 68% yield.

Analytics

MP: ca. 200° C. (decomposition).

H₂O content: 0.38%.

### Example 3

### 1-[2-(4-Benzyl-4-hydroxy-piperidin-1-yl)-ethyl]-3-(2-methyl-quinolin-4-yl)-urea sulfate

To a suspension of 1-[2-(4-benzyl-4-hydroxy-piperidin-1-yl)-ethyl]-3-(2-methyl-quinolin-4-yl)-urea of formula I (21.36 kg) in CH₃OH (178 L) is added aqueous H₂SO₄ (6 L, 9.91%) during 10 min. The clear solution is filtered and further aqueous H₂SO₄ (33.8 L, 1.07 M) is added during 45 min. The solution is cooled to −2° C. during 1.5 h and stirred at −5 to −9° C. for 1 h. The formed precipitate is filtered, washed with cooled CH₃OH (−5° C., 54 L) and dried under a stream of nitrogen to provide the title compound as white crystalline powder in 84% yield.

Analytics

H₂O content: 0.84%.

### Example 4

### 1-[2-(4-Benzyl-4-hydroxy-piperidin-1-yl)-ethyl]-3-(2-methyl-quinolin-4-yl)-urea sulfate

To a suspension of 1-[2-(4-benzyl-4-hydroxy-piperidin-1-yl)-ethyl]-3-(2-methyl-quinolin-4-yl)-urea of formula I (68.29 mol) in CH₃OH (285 L, 0.24 M solution of compound 1) is added aqueous H₂SO₄ (11 L, 9.91%) during 10 min. The clear solution is filtered and further aqueous H₂SO₄ (59.5 L, 9.91%, 1.07 M) is added during 30 min. The solution is cooled to −7° C. during 2 h and stirred at this temperature for 1 h. The formed precipitate is filtered, washed with cooled CH₃OH (−4° C., 41 L) and dried under a stream of nitrogen to provide the title compound as white crystalline powder in 83% yield.

Analytics

H₂O content: 0.58%.

¹H-NMR (D₂O): 7.97 (d, J=8.5, 1H); 7.75 (s, 1H); 7.65 (t, J=7.4, 1H); 7.53 (d, J=8.2, 1H); 7.45 (t, J=7.7, 1H); 7.21-7.07 (m, 5H); 3.62 (t, =5.7, 2H); 3.41-3.45 (m, 2H); 3.27 (t, J=-5.7, 2H); 3.08-3.16 (m, 2H); 2.68 (s, 2H); 2.54 (s, 3H); 1.88-1.93 (m, 2H); 1.67-1.71 (m, 2H).

### Example 5

### 1-[2-(4-Benzyl-4-hydroxy-piperidin-1-yl)-ethyl]-3-(2-methyl-quinolin-4-yl)-urea malate

A suspension of 1-[2-(4-benzyl-4-hydroxy-piperidin-1-yl)-ethyl]-3-(2-methyl-quinolin-4-yl)-urea of formula I (2.09 g, 0.005 mol) in acetone (50 mL) is heated at 50° C. and an aqueous solution of L-(−)-malic acid (738 mg in 10 mL) is added. The clear solution is cooled at 4° C. for 15 h. The formed precipitate is filtered, washed with acetone (20 mL) and dried in HV at 50° C. to provide the title compound as white crystalline powder in 71% yield.

Analytics

MP: 143-146° C. (decomposition).

H₂O content: 1.92%.

Elemental Analysis for C₂₉H₃₆N₄O₇ (0.60 H₂O): % found (calculated): C: 61.53 (61.82); H 6.60 (6.65); N 9.87 (9.94).

¹H-NMR (d6-DMSO): 9.12 (br. s, 1H); 8.12 (d, J=8.3, 1H); 8.07 (s, 1H); 7.82 (d, J=8.6, 1H); 7.65 (t, J≈7.2, 1H); 7.49 (t, J≈7.1, 1H); 7.27-7.15 (m, 5H); 7.08 (br. t, J≈4.6, 1 H); 4.49 (br. s, 1H); 4.05 (dd, J=5.9, 7.3, 1 H); 3.38 (m, 2H); 2.96 (m, 2H); 2.81 (m, 2H); 2.70 (m, 4H); 2.55 (dd, J=7.5, 15.5, 1H); 2.54 (s, 3H); 2.36 (dd, J=5.9, 15.6, 1H); 1.69-1.61 (m, 2H); 1.51-1.47 (m, 2H).

### Example 6

### 1-[2-(4-Benzyl-4-hydroxy-piperidin-1-yl)-ethyl]-3-(2-methyl-quinolin-4-yl)-urea citrate

A solution of citric acid (1.05 g) in C₂H₅OH (400 mL) is heated at 65° C. and 1-[2-(4-benzyl-4-hydroxy-piperidin-1-yl)-ethyl]-3-(2-methyl-quinolin-4-yl)-urea of formula I (2.09 g, 0.005 mol) is added portion wise as a solid. The mixture is stirred at 4° C. for 15 h. The formed precipitate is filtered and dried in HV at 50° C. to provide the title compound as white crystalline powder in 72% yield.

Analytics

MP: 152-157° C. (decomposition).

H₂O content: 1.08%.

Elemental Analysis for C₃₁H₃₈N₄O₉ (0.37 H₂O): % found (calculated): C: 60.51 (60.31); H 6.31 (6.33); N 8.97 (9.08).

¹H-NMR (CD₃OD): 8.46 (d, 8.5, 1H); 8.18 (s, 1H); 7.83 (d, J=7.9, 1H); 7.72 (t, J≈7.4, 1H); 7.57 (t, J=7.3, 1H); 7.28-7.15 (m, 5H); 3.68 (m, 2H); 3.48 (m, 2H); 3.31 (m, 2H); 3.17 (m, 2H); 2.87 (d, J=15.2, 2H), 2.80 (s, 2H); 2.70 (d, J=15.5, 2H), 2.67 (s, 3H); 2.07 (m, 2H); 1.68 (m, 2H).

The following examples serve to aid the understanding of the present invention.

### Example 7

### X-ray Diffraction Pattern (XRD)

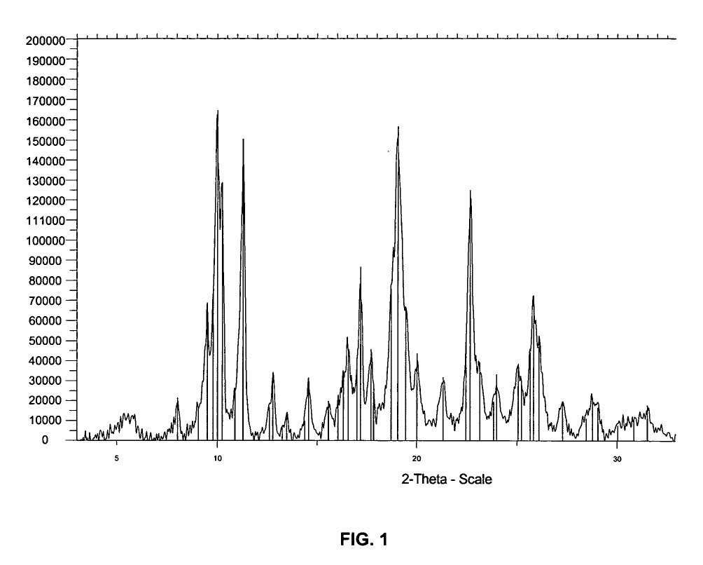

FIG. 1 shows the XRD-diffraction pattern of the compound described in Example 2. Table 1 summarizes the peaks and their intensity. It has to be understood that due to small changes in the experimental details, small deviations in the 2-Theta values of the characteristic peaks in the X-ray powder diffraction patterns may occur.

Example 2 Type: 2Th/Th locked—Start: 3.000°—End: 40.000°—Step: 0.020°—Step time: 1 s—Temp.: 30° C.—Time Started: 3 s—2-Theta: 3.00

DIF—Y: 97.92% d×by: 1.—WL: 1.54056—0—

TABLE 1
2-Theta Angles and their intensities of the X-ray diffraction
pattern (XRD) of the compound described in Example 2

| 2-Theta | | | |
|---|---|---|---|
| Angle | d value | Intensity | Intensity |
| [°] | [Angstrom] | [Count] | [%] |
| 7.986 | 11.062 | 21125 | 12.8 |
| 9.016 | 9.8 | 15544 | 9.4 |
| 9.46 | 9.341 | 68555 | 41.6 |
| 9.749 | 9.065 | 72939 | 44.3 |
| 9.974 | 8.861 | 164612 | 100 |
| 10.198 | 8.667 | 128740 | 78.2 |
| 10.836 | 8.158 | 25110 | 15.3 |
| 11.236 | 7.868 | 150662 | 91.5 |
| 12.562 | 7.041 | 16342 | 9.9 |
| 12.736 | 6.945 | 31886 | 19.4 |
| 13.217 | 6.693 | 5979 | 3.6 |
| 13.43 | 6.587 | 12356 | 7.5 |
| 14.339 | 6.172 | 9566 | 5.8 |
| 14.541 | 6.087 | 30690 | 18.6 |
| 15.538 | 5.698 | 19530 | 11.9 |
| 16.012 | 5.53 | 21922 | 13.3 |
| 16.289 | 5.437 | 32285 | 19.6 |
| 16.541 | 5.355 | 45039 | 27.4 |
| 16.979 | 5.218 | 41452 | 25.2 |
| 17.16 | 5.163 | 86491 | 52.5 |
| 17.674 | 5.014 | 45438 | 27.6 |
| 17.807 | 4.977 | 24313 | 14.8 |
| 18.687 | 4.744 | 75729 | 46 |
| 19.037 | 4.658 | 156640 | 95.2 |
| 19.429 | 4.565 | 65366 | 39.7 |
| 19.983 | 4.44 | 43445 | 26.4 |
| 21.284 | 4.171 | 31487 | 19.1 |
| 22.431 | 3.96 | 48626 | 29.5 |
| 22.641 | 3.924 | 124754 | 75.8 |
| 23.121 | 3.844 | 39060 | 23.7 |
| 23.812 | 3.734 | 19132 | 11.6 |
| 23.946 | 3.713 | 33082 | 20.1 |
| 25.033 | 3.554 | 38263 | 23.2 |
| 25.209 | 3.53 | 29893 | 18.2 |
| 25.641 | 3.471 | 44242 | 26.9 |
| 25.804 | 3.45 | 72541 | 44.1 |
| 26.081 | 3.414 | 52213 | 31.7 |
| 27.27 | 3.268 | 19132 | 11.6 |
| 28.436 | 3.136 | 10762 | 6.5 |
| 28.756 | 3.102 | 21125 | 12.8 |
| 29.023 | 3.074 | 16342 | 9.9 |
| 30.028 | 2.973 | 7174 | 4.4 |
| 30.824 | 2.898 | 10363 | 6.3 |
| 31.509 | 2.837 | 17537 | 10.7 |
| 33.925 | 2.64 | 13153 | 8 |
| 34.236 | 2.617 | 7573 | 4.6 |
| 34.803 | 2.576 | 9964 | 6.1 |
| 35.636 | 2.517 | 11160 | 6.8 |
| 36.094 | 2.486 | 7174 | 4.4 |
| 36.625 | 2.452 | 7174 | 4.4 |
| 37.882 | 2.373 | 5979 | 3.6 |
| 39.627 | 2.272 | 13552 | 8.2 |

### Example 8

### Hygroscopicity

Hygroscopicity was evaluated using the static method according to European Pharmacopoeia Technical Guide. Weight increase of the compound was observed when stored in a humidity cabinet at RT/79% RH for 24 h. The results are shown in Table 2.

TABLE 2
Increase in weight (Δm) after exposure to 79% RH for 24 h

| Example | Δm [%] |
|---|---|
| Compound I | 8 |
| as free base | |
| 2 | 4 |
| 5 | 5 |
| 6 | 2 |

### Example 9

### Solubility in Water and at pH 7

Solubility was measured in water and aqueous phosphate buffer (pH=7, 100 mM). Results are expressed as mg dissolved compound per mL solvent. The results are summarized in Table 3.

TABLE 3
Solubility in water and phosphate buffer

| | | Buffer pH 7 |
|---|---|---|
| | Solubility water | (phosphate, 100 mM) |
| Example | (mg/mL) | (mg/mL) |
| Compound I | 0.8 | 0.31 |
| 2 | 11.3 | 27 |
| 5 | >101 | — |
| 6 | 17.8 | — |

### Example 10

### Pharmacokinetic Assessment of 1-[2-(4-benzyl-4-hydroxy-piperidin-1-yl)-ethyl]-3-(2-methyl-quinolin-4-yl)-urea compounds

The pharmacokinetic parameters after oral (gavage) administration of 10 mg per kg of 1-[2-(4-benzyl-4-hydroxy-piperidin-1-yl )-ethyl]-3-(2-methyl-quinolin-4-yl )-urea given as free base or sulfate salt have been determined in male Wistar rats. Blood samples were taken over a time period of 24 h after dosing and analysed with a specific and sensitive liquid chromatography-mass-spectrometry (LC-MS/MS) method. Pharmacokinetic parameters were calculated using a non-compartmental method. The mean exposure of 1-[2-(4-benzyl-4-hydroxy-piperidin-1-yl)-ethyl]-3-(2-methyl-quinolin-4-yl)-urea, expressed as area under the curve (AUC_{0-inf.}), after administration of compound I as free base was 194 ng*h/mL. The mean exposure of 1-[2-(4-benzyl-4-hydroxy-piperidin-1-yl )-ethyl]-3-(2-methyl-quinolin-4-yl )-urea expressed as area under the curve (AUC_{0-inf.}), after administration of the compound described in Example 1 was 396 ng*h/mL.

1. The compound, 1-[2-(4-Benzyl-4-hydroxy-piperidin-1-yl)-ethyl]-3-(2-methyl-quinolin-4-yl)-urea of formula I in the form of a crystalline salt or non-defined crystalline salt hydrate.

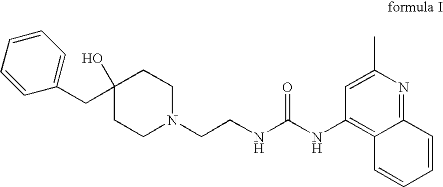

2. 1-[2-(4-Benzyl-4-hydroxy-piperidin-1-yl)-ethyl]-3-(2-methyl-quinolin-4-yl)-urea according to claim 1, said compound being in the form of a sulfate or a non-defined sulfate hydrate.

3. 1-[2-(4-Benzyl-4-hydroxy-piperidin-1-yl)-ethyl]-3-(2-methyl-quinolin-4-yl)-urea according to claim 1, said compound being in the form of a malate or a non-defined malate hydrate.

4. 1-[2-(4-Benzyl-4-hydroxy-piperidin-1-yl)-ethyl]-3-(2-methyl-quinolin-4-yl)-urea according to claim 1, said compound being in the form of a citrate or a non-defined citrate hydrate.

5. 1-[2-(4-Benzyl-4-hydroxy-piperidin-1-yl)-ethyl]-3-(2-methyl-quinolin-4-yl)-urea of formula I as a sulfate salt according to claim 1 having a corresponding X-ray powder diffraction pattern as depicted in FIG. 1.

6. 1-[2-(4-Benzyl-4-hydroxy-piperidin-1-yl)-ethyl]-3-(2-methyl-quinolin-4-yl)-urea of formula I as a sulfate salt according to claim 1 which shows peaks at the diffraction (2-theta) angles shown in the following table in its X-ray powder diffraction pattern.

| 2-Theta | | | |
|---|---|---|---|
| Angle | d value | Intensity | Intensity |
| [°] | [Angstrom] | [Count] | [%] |
| 7.986 | 11.062 | 21125 | 12.8 |
| 9.016 | 9.8 | 15544 | 9.4 |
| 9.46 | 9.341 | 68555 | 41.6 |
| 9.749 | 9.065 | 72939 | 44.3 |
| 9.974 | 8.861 | 164612 | 100 |
| 10.198 | 8.667 | 128740 | 78.2 |
| 10.836 | 8.158 | 25110 | 15.3 |
| 11.236 | 7.868 | 150662 | 91.5 |
| 12.562 | 7.041 | 16342 | 9.9 |
| 12.736 | 6.945 | 31886 | 19.4 |
| 13.217 | 6.693 | 5979 | 3.6 |
| 13.43 | 6.587 | 12356 | 7.5 |
| 14.339 | 6.172 | 9566 | 5.8 |
| 14.541 | 6.087 | 30690 | 18.6 |
| 15.538 | 5.698 | 19530 | 11.9 |
| 16.012 | 5.53 | 21922 | 13.3 |
| 16.289 | 5.437 | 32285 | 19.6 |
| 16.541 | 5.355 | 45039 | 27.4 |
| 16.979 | 5.218 | 41452 | 25.2 |
| 17.16 | 5.163 | 86491 | 52.5 |
| 17.674 | 5.014 | 45438 | 27.6 |
| 17.807 | 4.977 | 24313 | 14.8 |
| 18.687 | 4.744 | 75729 | 46 |
| 19.037 | 4.658 | 156640 | 95.2 |
| 19.429 | 4.565 | 65366 | 39.7 |
| 19.983 | 4.44 | 43445 | 26.4 |
| 21.284 | 4.171 | 31487 | 19.1 |
| 22.431 | 3.96 | 48626 | 29.5 |
| 22.641 | 3.924 | 124754 | 75.8 |
| 23.121 | 3.844 | 39060 | 23.7 |
| 23.812 | 3.734 | 19132 | 11.6 |
| 23.946 | 3.713 | 33082 | 20.1 |
| 25.033 | 3.554 | 38263 | 23.2 |
| 25.209 | 3.53 | 29893 | 18.2 |
| 25.641 | 3.471 | 44242 | 26.9 |
| 25.804 | 3.45 | 72541 | 44.1 |
| 26.081 | 3.414 | 52213 | 31.7 |
| 27.27 | 3.268 | 19132 | 11.6 |
| 28.436 | 3.136 | 10762 | 6.5 |
| 28.756 | 3.102 | 21125 | 12.8 |
| 29.023 | 3.074 | 16342 | 9.9 |
| 30.028 | 2.973 | 7174 | 4.4 |
| 30.824 | 2.898 | 10363 | 6.3 |
| 31.509 | 2.837 | 17537 | 10.7 |
| 33.925 | 2.64 | 13153 | 8 |
| 34.236 | 2.617 | 7573 | 4.6 |
| 34.803 | 2.576 | 9964 | 6.1 |
| 35.636 | 2.517 | 11160 | 6.8 |
| 36.094 | 2.486 | 7174 | 4.4 |
| 36.625 | 2.452 | 7174 | 4.4 |
| 37.882 | 2.373 | 5979 | 3.6 |
| 39.627 | 2.272 | 13552 | 8.2 |

7. A process for preparing 1-[2-(4-benzyl-4-hydroxy-piperidin-1-yl)-ethyl]-3-(2-methyl-quinolin-4-yl)-urea of formula I as a salt according to any one of claims 1 to 6, which process comprises
a. mixing 1-[2-(4-benzyl-4-hydroxy-piperidin-1-yl)-ethyl]-3-(2-methyl-quinolin-4-yl)-urea of formula I with an organic solvent and adding an acid, a solution of an acid in water, a solution of an acid in an organic solvent, or a solution of an acid in a mixture of water with an organic solvent, and stirring the mixture; or
b. mixing 1-[2-(4-benzyl-4-hydroxy-piperidin-1-yl)-ethyl]-3-(2-methyl-quinolin-4-yl)-urea of formula I with a mixture of an organic solvent with water and adding an acid, a solution of an acid in water, a solution of an acid in an organic solvent, or a solution of an acid in a mixture of water with an organic solvent, and stirring the mixture; or
c. adding 1-[2-(4-benzyl-4-hydroxy-piperidin-1-yl )-ethyl]-3-(2-methyl-quinolin-4-yl)-urea of formula I as a solid, or dissolved in a mixture of an organic solvent with water to an acid, to a solution of an acid in water, to a solution of an acid in an organic solvent, or to a solution of an acid in a mixture of water with an organic solvent, and stirring the mixture; or
d. adding 1-[2-(4-benzyl-4-hydroxy-piperidin-1-yl)-ethyl]-3-(2-methyl-quinolin-4-yl)-urea of formula I as a solid, or dissolved in an organic solvent to an acid, to a solution of an acid in water, to a solution of an acid in an organic solvent, or to a solution of an acid in a mixture of water with an organic solvent, and stirring the mixture.

8. A process for preparing 1-[2-(4-benzyl-4-hydroxy-piperidin-1-yl)-ethyl]-3-(2-methyl-quinolin-4-yl)-urea of formula I as a salt, comprising:
a. mixing 1-[2-(4-benzyl-4-hydroxy-piperidin-1-yl)-ethyl]-3-(2-methyl-quinolin-4-yl)-urea of formula I with an organic solvent and adding an acid, a solution of an acid in water, a solution of an acid in an organic solvent, or a solution of an acid in a mixture of water with an organic solvent, and stirring the mixture; or
b. mixing 1-[2-(4-benzyl-4-hydroxy-piperidin-1-yl)-ethyl]-3-(2-methyl-quinolin-4-yl)-urea of formula I with a mixture of an organic solvent with water and adding an acid, a solution of an acid in water, a solution of an acid in an organic solvent, or a solution of an acid in a mixture of water with an organic solvent, and stirring the mixture; or
c. adding 1-[2-(4-benzyl-4-hydroxy-piperidin-1-yl)-ethyl]-3-(2-methyl-quinolin-4-yl)-urea of formula I as a solid, or dissolved in a mixture of an organic solvent with water to an acid, to a solution of an acid in water, to a solution of an acid in an organic solvent, or to a solution of an acid in a mixture of water with an organic solvent, and stirring the mixture; or
d. adding 1-[2-(4-benzyl-4-hydroxy-piperidin-1-yl)-ethyl]-3-(2-methyl-quinolin-4-yl)-urea of formula I as a solid, or dissolved in an organic solvent to an acid, to a solution of an acid in water, to a solution of an acid in an organic solvent, or to a solution of an acid in a mixture of water with an organic solvent, and stirring the mixture.

9. 1-[2-(4-Benzyl-4-hydroxy-piperidin-1-yl)-ethyl]-3-(2-methyl-quinolin-4-yl)-urea of formula I as a salt obtained by the process of claim 8.

10. A composition comprising 1-[2-(4-benzyl-4-hydroxy-piperidin-1-yl)-ethyl]-3-(2-methyl-quinolin-4-yl)-urea of formula I as a salt according to any one of claims 1 to 6 and inert carrier material.

11. A pharmaceutical composition containing one or more compounds of any one of claims 1 to 6, inert carrier material and/or an adjuvant.

12. The pharmaceutical composition of claim 11 further comprising one or more additional pharmacologically active compounds.

13. The pharmaceutical composition of claim 12 wherein one or more additional pharmacologically active compounds are selected from the group consisting of ACE inhibitors, angiotensin II receptor antagonists, endothelin receptor antagonists, vasopressin antagonists, beta-adrenergic antagonists, alpha-adrenergic antagonists, vasopressin antagonists, TNFalpha antagonists, or peroxisome proliferator activator receptor modulators.

14. A method of treating a patient suffering from a disorder associated with dysregulation of urotensin II or urotensin II receptors, or a disorder associated with vascular or myocardial dysfunction, comprising administering the pharmaceutical composition according to claim 11.

15. The method of claim 14, wherein the disorder is selected from the group consisting of hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension and pulmonary fibrosis.

16. A method of treating or preventing a disorder or disease, comprising administering the pharmaceutical composition of claim 11, wherein the disorder or disease is associated with restenosis after balloon or stent angioplasty, or is selected from the group consisting of cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, diabetic complications, complications of vascular or cardiac surgery or organ transplantation, complications of cyclosporin treatment, pain, addictions, schizophrenia, Alzheimer's disease, anxiety, obsessive-compulsive behavior, epileptic seizures, stress, depression, dementias, neuromuscular disorders and neurodegenerative diseases.

17. A method of treating a disorder associated with dysregulation of urotensin II or urotensin II receptors, or a disorder associated with vascular or myocardial dysfunction, comprising administering the pharmaceutical composition of claim 12.

18. The method of claim 17, wherein the disorder is selected from the group consisting of hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension and pulmonary fibrosis.

19. A method of treating or preventing a disorder or disease, comprising administering the pharmaceutical composition of claim 12, wherein the disorder or disease is associated with restenosis after balloon or stent angioplasty, or is selected from the group consisting of cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, diabetic complications, complications of vascular or cardiac surgery or organ transplantation, complications of cyclosporin treatment, pain, addictions, schizophrenia, Alzheimer's disease, anxiety, obsessive-compulsive behavior, epileptic seizures, stress, depression, dementias, neuromuscular disorders and neurodegenerative diseases.

20. A method of treating a disorder associated with dysregulation of urotensin II or urotensin II receptors, or a disorder associated with vascular or myocardial dysfunction, comprising administering the pharmaceutical composition of claim 13.

21. The method of claim 20, wherein the disorder is selected from the group consisting of hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension and pulmonary fibrosis.

22. A method of treating or preventing a disorder or disease, comprising administering the pharmaceutical composition of claim 13, wherein the disorder or disease is associated with restenosis after balloon or stent angioplasty, or is selected from the group consisting of cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, diabetic complications, complications of vascular or cardiac surgery or organ transplantation, complications of cyclosporin treatment, pain, addictions, schizophrenia, Alzheimer's disease, anxiety, obsessive-compulsive behavior, epileptic seizures, stress, depression, dementias, neuromuscular disorders and neurodegenerative diseases.

23. A composition comprising a crystalline part and an amorphous part of 1-[2-(4-benzyl-4-hydroxy-piperidin-1-yl)-ethyl]-3-(2-methyl-quinolin-4-yl )-urea of formula I as a salt according to any one of claims 1 to 6.
